(19) United States
(12) Patent Application Publication
(10) Pub. No.: US 2004/0037903 A1
(43) Pub. Date: Feb. 26, 2004

(54) Phytonutrient formula for the relief of chronic pain resulting from inflammation

(76) Inventors: Edward A. Lemmo, Staten Island, NY (US); Risa N. Schulman, Los Angeles, CA (US)

Correspondence Address: Paul F Fehlner, Darby & Darby, Post Office Box 5257, New York, NY, 10150-5257, US

(21) Appl. No.: 10/297,896

(22) PCT Filed: Jun. 13, 2001
(86) PCT No.: PCT/US01/18989

(51) Int. Cl. A61K 35/78; A61K 31/05

(52) U.S. Cl. 424/756; 514/733; 424/774

(57) ABSTRACT

The present invention is a dietary supplement composition comprising resveratrol and turmeric extract. The composition may further contain other components including stinging nettle, and Boswellia serrata. The composition is useful due to its properties as an anti-inflammatory, analgesic, cartilage protectant and antioxidant agent.

### FIELD OF THE IVENTION

The present invention relates generally to dietary supplement compositions, and more particularly to a composition comprising resveratrol and turmeric extract. The claimed dietary supplement possesses anti-inflammatory, analgesic, cartilage protectant and antioxidant properties.

### BACKGROUND OF THE INVENTION

Inflammation and various inflammatory disorders are highly prevalent, afflicting large percentages of the world's population. Inflammation is a necessary response of the body to counteract the threat of infectious agents and other foreign bodies, and is characterized by symptoms such as pain, redness, heat, swelling and decreased function. These symptoms are caused by a variety of inflammatory mediators acting in concert, such as prostaglandins, thromboxane, and leukotrienes. Overexpression of these factors is implicated in a variety of conditions, particularly rheumatoid arthritis, an autoimmune disease characterized by chronic inflammation and cartilage destruction.

Two classes of drugs have been used to combat pain and inflammation. Salicylic acid and related compounds inhibit prostaglandins by acting on the cyclooxygenase pathway. Glucocorticosteroids have been used to inhibit both the cyclooxygenase pathway and the lipooxygenase pathways by interfering with the phospholipase A2-reaction. Although usage of these anti-inflammatory drugs is common, their administration over a long period of time has well known side effects. Therefore, the need to explore alternative anti-inflammatory strategies using non-toxic natural products is warranted. Though a large number of dietary supplements are currently known and marketed, no such supplements include a novel mixture of phytonutrients that provides a safe, non-toxic, anti-inflammatory and analgesic effect.

It is therefore an object of the present invention to provide a non-toxic supplement which exhibits an anti-inflammatory and analgesic effect without the known deleterious side effects associated with compositions within the prior art.

It is also an object of the present invention to provide a composition, which in addition to its anti-inflarnmatory effect, is useful as an analgesic, a cartilage protectant and/or an antioxidant.

### SUMMARY OF THE INVENTION

The present invention is directed to a dietary supplement composition comprising resveratrol and turmeric extract. The claimed dietary supplement is useful as a nutritional supplement, anti-inflammatory, analgesic, cartilage protectant and/or antioxidant.

The present invention further provides a method for treating, ameliorating or preventing inflammation, pain, cartilage deterioration or oxidative degradation through the administration of a dietary supplement composition comprising resveratrol and turmeric extract.

### DETAILED DESCRIPTION OF THE INVENTION

The present invention provides a dietary supplement composition comprising resveratrol and turmeric extract. The claimed dietary supplement is useful as a nutritional supplement, anti-inflammatory, analgesic, cartilage protectant and/or antioxidant.

The present invention further provides a method for treating, ameliorating or preventing inflammation, pain, cartilage deterioration or oxidative degradation through the administration of a dietary supplement composition comprising resveratrol and turmeric extract.

Preferably, the claimed dietary supplement composition further comprises stinging nettle and/or Boswellia serrata.

The resveratrol and turmeric extract, as well as the optional stinging nettle and Boswellia serrata ingredients, inhibit the enzymes cyclooxygenase and 5-lipoxygenase and/orthe production of their products, prostaglandins (PG), leukotrienes (LT), and thromboxanes (TX). Through such inhibition, they exhibit anti-inflammatory and analgesic effects. The body's production of other molecules mediating the production of these enzyme products is also blocked, such as interleukins (IL) and tumor necrosis factor (TNF). Turmeric further acts as an antioxidant, complementing these anti-inflammatory and analgesic effects. Both Boswellia serrata and turmeric further exert cartilage protectant properties, thereby acting in the areas of the joints, a prime site for pain and/or inflammation to arise.

The claimed composition comprises about 1 to about 4000 mg of resveratrol and about 1 mg to about 4000 mg of turmeric extract. Preferably, the claimed composition comprises from about 10 mg to about 400 mg of resveratrol and about 50 mg to about 450 mg of turmeric extract. Still more preferably, such composition comprises from about 15 mg to about 45 mg of resveratrol. Most preferably, the claimed composition comprises about 30 mg of resveratrol, and about 50 mg of turmeric extract.

In a preferred embodiment of the invention, the claimed composition comprises about 1 to about 4000 mg of resveratrol, about 1 mg to about 4000 mg of turmeric extract, about 1 mg to about 4000 mg of stinging nettle and about 1 mg to about 4000 mg of Boswellia serrata. Preferably, such composition comprises about 10 to about 400 mg of resveratrol (more preferably about 15 to about 45 mg), about 50 mg to about 450 mg of turmneric extract, about 50 mg to about 400 mg of stinging nettle and about 100 mg to about 400 mg of Boswellia serrata. Most preferably, such composition comprises about 30 mg of resveratrol, about 50 mg of turmeric extract, about 50 mg of stinging nettle and about 200 mg of Boswellia serrata.

In order to administer consistent quantities of compounds of biological origins, many biologically-derived compounds are now “standardized”. This process involves first analyzing the select chemical components of a biological material and quantitatively evaluating its components. A specific component is then selected as the “marker compound”. Once the ratios between various components and the marker compound are known, analyzing a proposed sample for the marker compound allows one to determine the relative quantities of the samnple's other components. The marker compound is the compound against which a given material is said to be “standardized”. For example, the composition claimed herein utilizes stinging nettles as a component. Most preferably, the type of this component employed in the practice of the invention is one that is standardized to “1% of silicic acid”. This means that an extract of stinging nettles concentrated to contain 1% of silicic acid is preferably used.

Resveratrol Component

Resveratrol (3,5,4′-trihydroxy-trans-stilbene) is a ubiquitous phytoalexin synthesized by numerous plant species. Since relatively high quantities are found in grape skin (Vitis vinifera) and red wines, resveratrol is routinely incorporated into the average diet. Resveratrol has been proposed to alter the production of eicosanoids, a family of molecules involved in inflammation, pain perception and immune response, including prostaglandins (PGs), thromboxanes (TXs) and leukotrienes (LTs).

The arachidonic acid pathway is responsible for the production of eicosanoids. Arachidonic acid is the starting compound for two different pathways, one mediated by cyclooxygenase enzymes (i.e. COX-2) and one by lipoxygenase enzymes (i.e. 5-LOX). The arachidonic acid pathway is also mediated by a group of molecules called cytokines, which include interleukins (ILs) and tumor necrosis factor (TNF-α).

Resveratrol is a potent inhibitor of both the cyclooxygenase (COX) and lipoxygenase (LOX) pathways. There are two isoforms of COX, referred to as COX-1 and COX-2, which are key enzymes that catalyze the biosynthesis of eicosanoids such as prostaglandins. Non-steroidal anti-inflammatory drugs (NSAIDs) act by inhibiting both COX-1 and COX-2. Inhibition of COX-1 is the basis for the adverse side effects associated with prolonged use of NSAIDs. Resveratrol inhibits COX-2 only, and therefore does not cause these side effects, but retains the anti-inflammatory and analgesic actions.

Studies have shown that resveratrol not only inhibits the COX-2 enzyme directly, but also completely blocks its synthesis as represented by decreased levels of COX-2 protein, mRNA and gene promoter activity. The involved region of the COX-2 promoter has been determined. Resveratrol also completely inhibited signaling molecules protein kinase C, c-Jun, ERK1 and AP-1, effectively blocking the signal transduction pathway necessary for the induction of the COX-2 geneProduction of many of the major eicosanoids is thereby inhibited, including COX-2 products PGE₂ (prostaglandin E2), PGF₂ (prostaglandin F2), PGD₂ (prostaglandin D2), TXB₂ (thromboxane B2) and HHT (12-hydroxy-5,8,10-heptadecatrienoic acid), and 5-LOX products 5-HETE (5-hydroxy-6,8,11,14-eicosatetraenoic acid-), 5,12-diHETE (5,12-hydroxy-6,8,11,14-eicosatetraenoic acid)and LTC₄ (leukotriene C4).

It was recently reported that resveratrol also has an effect on cytokines. Incubation of mouse macrophages with resveratrol inhibited the production of the IL-6 by 34%.

An additional effect of resveratrol is its ability to inhibit the induction of inducible nitric oxide synthase (iNOS). iNOS mediates the synthesis of nitric oxide, which modulates a range of physiological responses including inflammation.

Leukotrienes, products of the LOX pathway, are involved in immunoregulation and in a variety of diseases, including asthma, inflammation and various allergic conditions. In the presence of 5-lipoxygenase, free arachidonic acid is converted into 5-hydroperoxy-6,8,11,14-eicosatetraenoic acid (5-HPETE) which is then reduced to 5-hydroxy-6,8,11,14-eicosatetraenoic acid (5-HETE) or dehydrated to the unstable intermediate leukotriene A4 (LTA4) which can be converted enzymatically to LTB4, LTC4 and LTD4. Resveratrol strongly inhibits the formation of 5-HETE. In rat polymorphonuclear leukocytes, 5-HETE was inhibited by greater than 70%. Furthermore, LOX products 5,12-diHETE, 5-HETE and LTC4 were also inhibited. Resveratrol also stimulates cAMP levels, dose dependently. It is well accepted that cAMP plays an important role in inflammatory events. It is thought that cAMP is involved in the control of a variety of inflammatory and immunologic processes, such as the release of histamine, leukotrienes and lysosomal enzymes.

The ability of resveratrol to inhibit eicosanoid production catalyzed by COX-2 and 5-LOX provides a mechanistic rationale for the anti-inflammatory and analgesic action of this compound.

Resveratrol is commercially available. For example, it is sold by InterHealth Nutraceuticals, Inc. of Benicia, Calif., under the tradename Protykin®.

Turmeric Component

Experimentally, turmeric (Curcuma longa) has been reported to possess anti-inflammatory, anticarcinogenic and free radical scavenging properties in animals. The active component isolated from turmeric is curcumin (diferuloyl methane), which is responsible for its color and flavor. Studies show curcumin has a potent effect on the inflammatory response. Curcumin directly blocks the activity of COX-2 and 5-LOX in rat and human cell lines. Curcumin also inhibits the synthesis of new COX-2 protein and mRNA. It has further been demonstrated that products of these pathways are also reduced, including PGE2, LTB4 and LTC4. These results were also seen in vivo in rats where PGE2 was reduced by 38% and 5-HETE by 80% with addition of curcumin to the diet. The cytokines IL-1B, IL-8 and TNF-α were also inhibited by at least 50% in human cell lines.

The mechanism by which curcumin affects these molecules is recently coming to light. The cytokines TNF-a and IL-1B activate NF-KB, a transcription factor that turns on multiple genes involved in inflammation. Studies have shown that curcumin blocks factors that remove the inhibitory molecule I-KBa from the NF-KB molecule, effectively keeping NF-KB in a dormant stage. As a result, genes encoding pro-inflammatory molecules are not turned on. It has been shown that production of IL-8 is blocked in this way.

The potency of turmeric has been evaluated in comparison to other anti-inflammatory drugs. In one human trial by Deodhar et al. (1980), the anti-inflammatory actions of curcumin were compared to phenylbutazone in patients with rheumatoid arthritis. Curcumin provided significant improvement in the duration of morning stiffness, walking time and joint swelling compared to phenylbutazone. Furthermore, curcumin has a lower ulcerogen index (0.6) than a similar active dose of phenylbutazone (1.7) based on an animal study.

The tumeric component useful in the practice of the present invention is commercially available. For example, a preferred standardized form of the tureric component, namely turmeric extract (95% curcuminoids), is commercially available from Sabinsa Corporation of Piscataway, N.J. under the tradename Curcumin C³ Complex.

Stinging Nettle Component

Stinging Nettle (Urtica dioica L.) is named for the fine silica-rich hairs that cover its stem and leaves and break off when touched. Nettle has been used for centuries as an herbal remedy for a variety of conditions. It has been shown that stinging nettle extracts inhibit the expression of several cytokines as well as eicosanoid formation in blood cells. Cytokines, in particular tumor necrosis factor (TNF), are elevated in the synovial fluid in arhritic conditions and are presumably involved in the disease process by up-regulating multiple inflammatory mediators. One of the substances acting early in the inflammatory cascade is transcription factor NF-κB, which causes the release of pro-inflammatory molecules IL-1, IL-2, IL-6, IL-8 and TNF-α. In many inflammatory diseases NF-κB activation is increased, leading to the over-expression of pro-inflammatory gene products. NF-κB is ubiquitously found as an inactive complex in the cytoplasm bound to its inhibitory sub-unit IκB. The extract of stinging nettle is known to markedly inhibit production of NF-κB in human cell lines. Research suggests that the extracts stabilizes the inhibitor IκB-α by preventing its proteolytic degradation. Stinging nettle also directly inhibits the production of TNF-α and IL-1β. Another transcription factor that has been implicated in the pathogenesis of rheumatoid artritis is activator protein-1 (AP-1). It has been suggested that increased AP-1 activation may be responsible for synovial hyperplasia in rheumatoid arthritis. Target genes of AP-1 include matrix metalloproteinases, which are involved in degrading connective tissues. AP-1 can physically interact with NF-κB and cooperatively induce cytokine gene expression. It has been demonstrated that leaf extract from stinging nettle potently inhibited AP-1.

Stinging nettle is commercially available. For example, the preferred standardized form of the component, namely stinging nettle (1% silicic acid), is sold by Pharmachem Laboratories, Inc. of Kearny, N.J., under the tradename P E Stinging Nettle 1%.

Boswellia serrata Component

The gum resin of Boswellia serrata (65% boswellic acid), or frankincense, is largely composed of pentacyclic triterpenes called boswellic acids (BAs). BAs have been shown to affect the activity of 5-lipoxygenase and leukocytes, both significantly involved in the inflammatory response. In vitro studies in human leukocytes and rat neutrophils have demonstrated that BAs specifically inhibit the synthesis of products of 5-lipoxygenase (LTB4, LTC4, 5,12-diHETE and 5-HETE) by greater than 80%. This effect is due to the binding of BAs to 5-lipoxygenase in a way that prevents the binding of its natural substrate, arachidonate. Additional studies showed that BAs dramatically decreased the migration and total count of leukocytes to arritic.

BAs have also been shown to have a positive effect on arthritic symptoms. In a 1996 study in rats, BAs prevented inflammation and arthritic activity in developing and established arthritis. Anti-inflammatory agents often decrease the content and synthesis of glycosaminoglycans, important components of cartilage degraded in arthritis. BAs reduce the synthesis, but not the total content of glycosaminoglycans, suggesting that BAs inhibit the destruction of cartilage. In support of this possibility, it has been found that BAs inhibit several cartilage-destructive enzymes, including leukocyte elastase and beta-glucuronidase. Pentacyclic triterpenes from sources other than Boswellia inhibit 5-lipoxygenase but do not additionally inhibit cartilage-destructive enzymes.

BAs are well tolerated. In acute, sub-acute and chronic toxicity studies with mice, rats and monkeys, no adverse effects or deaths were recorded. Even at doses of 1000 mg/kg, BAs had no ulcerogenic effects, as does aspirin.

The Boswellia serrata component useful in the practice of the current inveniton is commercially available. For example, the preferred standardized form of the Boswellia serrata, namely Boswellia serrata (65% boswellic acid), is commercially available from Sabinsa Corporation under the tradename Boswellin®.

The claimed composition may be administered in the form of pharmaceutically acceptable tablets, capsules, powder, paste, solutions, suspension or gel dosage forms. Preferably, tablets or capsules are used. Preferably, the route of administration is via the alimentary tract.

The claimed composition may be administered whenever inflammation or pain (acute or chronic) is experienced. The composition may further be taken to prevent or ameliorate inflammation, pain, cartilage deterioration or oxidative degradation.

Because the composition appears to be non-toxic, there is no known upper limit on the intake of the claimed composition. Because the components of the claimed composition are biologically derived, the dosages thereof will further vary with the concentration of the individual components therein.

Additional modifications and improvements of the present invention may also by apparent to those skilled in the art. Thus, the particular combination of parts described and illustrated herein is intended to represent only one embodiment of the invention, and is not intended to serve as limitations of alternative devices within the spirit and scope of the invention.

1. A composition comprising:
about 1 mg to about 4000 mg of resveratrol; and
about 1 mg to about 4000 mg of turmeric extract.

2. The composition of claim 1 comprising from about 10 to about 400 mg of resveratrol.

3. The composition of claim 1 comprising from about 15 to about 45 mg of resveratrol.

4. The composition of claim 1 comprising about 30 mg of resveratrol.

5. The composition of claim 1 comprising from about 1 to about 4000 mg of turmeric extract.

6. The composition of claim 1 comprising from about 50 to about 450 mg of turmeric extract.

7. The composition of claim 1 comprising about 50 mg of turmeric extract.

8. The composition of claim 5 wherein the turmeric extract is standardized to curcuminoids.

9. The composition of claim 1 comprising about 50 mg of standardized turmeric extract (95% curcuminoids).

10. The composition of claim 1 further comprising from about 1 to about 4000 mg of stinging nettle.

11. The composition of claim 10 comprising from about 50 to about 400 mg of stinging nettle.

12. The composition of claim 10 comprising about 50 mg of stinging nettle.

13. The composition of claim 10 wherein the stinging nettle is standardized to silicic acid.

14. The composition of claim 12 comprising about 50 mg of standardized stinging nettle (1% silicic acid).

15. The composition of claim 1 further comprising from about 1 to about 4000 mg of Boswellia serrata.

16. The composition of claim 15 comprising from about 100 to about 400 mg of Boswellia serrata.

17. The composition of claim 15 comprising about 200 mg of Boswellia serrata.

18. The composition of claim 15 wherein the Boswellia serrata is standardized against boswellic acid.

19. The composition of claim 18 comprising about 200 mg of standardized Boswellia serrata (65% boswellic acid).

20. The composition of claim 1 further comprising an amino acid, carbohydrate, lipid extract, oil, vitamin, mineral, botanical, botanical extract, nutraceutical, or phytonutrient.

21. A composition comprising:
about 1 mg to about 4000 mg of resveratrol;
about 1 mg to about 4000 mg of turmeric extract;
about 1 to about 4000 mg of stinging nettle; and
about 1 to about 4000 mg of Boswellia serrata

22. A composition comprising:
about 10 mg to about 400 mg of resveratrol;
about 50 mg to about 400 mg of turmeric extract;
about 50 to about 4000 mg of stinging nettle; and
about 100 to about 400 mg of Boswellia serrata.

23. A composition comprising:
about 15 mg to about 45 mg of resveratrol;
about 50 mg to about 400 mg of turmeric extract;
about 50 to about 4000 mg of stinging nettle; and
about 100 to about 400 mg of Boswellia serrata.

24. A composition comprising:
about 30 mg of resveratrol;
about 50 mg of standardized stinging nettle (1% silicic acid);
about 50 mg of standardized turmeric extract (95% curcuminoids);
about 200 mg of standardized Boswellia serrata (65% boswellic acid).

25. A method of treating, ameliorating or preventing inflammation, pain, cartilage deterioration or oxidative degradation comprising administering to a patient a composition comprising:
about 1 mg to 4000 mg of resveratrol; and
about 1 mg to 4000 mg of standardized turmeric extract.

26. The method of claim 25 wherein the composition comprises from about 10 to about 400 mg of resveratrol.

27. The method of claim 25 wherein the composition comprises from about 15 to about 45 mg of resveratrol.

28. The method of claim 25 wherein the composition comprises about 30 mg of resveratrol.

29. The method of claim 25 wherein the composition comprises from about 1 to about 4000 mg of turmeric extract.

30. The method of claim 25 wherein the composition comprises from about 50 to about 450 mg of turmeric extract.

31. The method of claim 25 wherein the composition comprises about 50 mg of turmeric extract.

32. The method of claim 25 wherein the turmeric extract is standardized to curcuminoids.

33. The method of claim 25 wherein the composition comprises about 50 mg of standardized turmeric extract (95% curcuminoids).

34. The method of claim 25 wherein the composition further comprises from about 1 to about 4000 mg of stinging nettle.

35. The method of claim 34 wherein the composition comprises from about 50 to about 400 mg of stinging nettle.

36. The method of claim 34 wherein the composition comprises about 50 mg of stinging nettle.

37. The method of claim 34 wherein the stinging nettle is standardized to silicic acid.

38. The method of claim 34 wherein the composition comprises about 50 mg of standardized stinging nettle (1% silicic acid).

39. The method of claim 25 wherein the composition further comprises from about 1 to about 4000 mg of Boswellia serrata.

40. The method of claim 39 wherein the composition comprises from about 100 to about 400 mg of Boswellia serrata.

41. The method of claim 39 wherein the composition comprises about 200 mg of Boswellia serrata.

42. The method of claim 39 wherein the Boswellia serrata is standardized against boswellic acid.

43. The method of claim 39 wherein the composition comprises about 200 mg of standardized Boswellia serrata (65% boswellic acid).

44. The method of claim 25 wherein the composition firther comprises an amino acid, carbohydrate, lipid extract, oil, vitamin, mineral, botanical, botanical extract, nutraceutical, or phytonutrient.

45. A method of treating, ameliorating or preventing inflammation, pain, cartilage deterioration or oxidative degradation comprising administering to a patient a composition comprising:
about 1 mg to about 4000 mg of resveratrol;
about 1 mg to about 4000 mg of turmeric extract;
about 1 to about 4000 mg of stinging nettle; and
about 1 to about 4000 mg of Boswellia serrata

46. The method of claim 45 wherein the composition comprises:
about 10 mg to about 400 mg of resveratrol;
about 50 mg to about 400 mg of turmeric extract;
about 50 to about 4000 mg of stinging nettle; and
about 100 to about 400 mg of Boswellia serrata.

47. The method of claim 45 wherein the composition comprises
about 15 mg to about 45 mg of resveratrol;
about 50 mg to about 400 mg of turmeric extract;
about 50 to about 4000 mg of stinging nettle; and
about 100 to about 400 mg of Boswellia serrata.

48. The method of claim 45 wherein the composition comprises:
about 30 mg of resveratrol;
about 50 mg of standardized stinging nettle (1% silicic acid);
about 50 mg of standardized turmeric extract (95% curcuminoids);
about 200 mg of standardized Boswellia serrata (65% boswellic acid).
